(19) United States
(12) Patent Application Publication
(10) Pub. No.: US 2011/0091445 A1
(43) Pub. Date: Apr. 21, 2011

(54) MODULATION OF INTESTINAL FLORA OF HIV PATIENTS

(75) Inventors: Jan Knol, Wageningen (NL); Monique Haarman, Wageningen (NL)

(73) Assignee: N.V. Nutricia, Zoetermeer (NL)

(21) Appl. No.: 12/666,077

(22) PCT Filed: Jun. 19, 2008
(86) PCT No.: PCT/NL08/50398

§ 371 Date: Nov. 15, 2010

(51) Int. Cl. A61K 39/395; A61K 31/715; A61K 31/732; A61P 31/04; A61P 31/10; A61P 37/02; A61P 1/00

(52) U.S. Cl. 424/130.1; 514/54

(57) ABSTRACT

The present invention is in the field of nutrition for HIV-infected subjects and concerns methods and composition comprising dietary fiber for the normalization of the intestinal flora of HIV-infected subjects.

### FIELD OF THE INVENTION

The present invention is in the field of nutrition for HIV-infected subjects.

### BACKGROUND OF THE INVENTION

Subjects, patients, infected with HIV suffer from many disease related symptoms several of which relate to the deterioration of the intestinal barrier function. A recent publication shows that circulating microbial products, probably derived from the gastrointestinal tract, are a cause of HIV-related systemic chronic immune activation. Circulating lipopolysaccharide, which was used as an indicator of microbial translocation, was significantly increased in HIV-infected individuals. [Brenchley J. M. et al. Nature Medicine (2006), 1365-1371]

Several mechanisms are known to modulate intestinal barrier function in HIV patients. WO 2005/122791 describes the use of a fatty acid composition for the modulation of HIV barrier function.

WO 2006/112717 discloses a composition comprising dietary fibres for the treatment and/or prevention of HIV. In WO 2006/112717 it is explained, see the summary of the invention and in particualr the last paragraph thereof, that “A healthy gut and healthy gut flora are intricately linked to healthy immune function.” It is specifically explained that dietary fibers influence the composition of Bifidobacteria and lactobacilli and that consequently beneficial immune effects occur and also that dietary fibers influence the functioning of the gut flora via fermentation of the fibers thereby producing compounds that influence general and immunological function of gut cells. The contribution WO 2006/112717 has over the prior art is the finding that the DC-SIGN molecule of dendritic cells can be blocked by certain oligosaccharides. It is explained that the blockage of DC-SIGN potentially prevents the transmission of HIV. WO 2006/112717 is silent on any potentially gut flora induced beneficial immune function. WO 2006/112717 does not disclose modulation of intestinal flora.

### SUMMARY OF THE INVENTION

The inventors surprisingly found that the intestinal bacterial flora in HIV-infected subjects is markedly changed compared to healthy persons. The relative amount of bifidobacteria (% of total fecal microbiota) was less than 5% of the total fecal microorganisms in HIV-infected subjects, whereas in healthy populations in general this level varies between 5 and 10%. Moreover it was surprisingly found that the numbers of bacterial pathogens e.g. Pseudomonas are increased in HIV-infected subjects.

Without being bound by theory, the inventors hypothesize that this change in flora contributes to the chronic activation of the immune system during progressive HIV disease and might result in deterioration of the intestinal barrier function. The development of a healthy intestinal flora is particularly important in HIV patients for the prevention and treatment of intestinal barrier disorders resulting not only in colic, congestion, diarrhea and bloody stools but also in chronic systemic immune activation which is a hallmark of progressive HIV infection. Surprisingly the present inventors found for the first time that the intestinal bacterial flora of HIV-infected subjects can be modulated by dietary fibres such that the number of bifidobacteria become similar by administering specific fibers to the HIV-infected subjects as in non-HIV patients.

Further, Pseudomonas is a pathogen that is hardly detectable in normal intestinal flora but has been demonstrated by the present inventors as being significantly increased in the intestinal flora of HIV patients (see the examples). The presence of potentially pathogenic amounts of Pseudomonas species is clearly not beneficial for immune compromised patients. Surprisingly, the inventors found that by administering specific fibers to HIV-infected subjects the numbers Pseudomonas in the intestinal flora decreased to levels normally found in healthy persons.

It is an object of the present invention to provide a method and a composition to normalize intestinal bacterial flora in HIV-infected subjects.

This object is met by a method for normalization of intestinal flora of HIV-infected subjects, said method comprising administering a composition comprising dietary fiber to said subject. Accordingly, also a composition is provided that is suitable for administering to HIV-infected subjects with the purpose of normalizing intestinal bacterial flora, that comprises dietary fiber.

In view of the present invention it is noted that from WO 2006/112717 it was not known that the intestinal flora of HIV was changed compared to healthy adults, neither was it known that pathogenic levels of Pseudomonas were present in the intestinal flora of HIV patients and that they could be treated with the administration of dietary fibers.

### DETAILED DESCRIPTION OF THE INVENTION

The present invention relates to a method for normalization of intestinal flora of HIV-infected subjects, said method comprising administering a composition comprising dietary fiber to said HIV-infected subject. For some jurisdictions the present invention can be worded as the use of a composition comprising dietary fiber for the preparation of a medicament or nutritional composition for normalizing intestinal bacterial flora in a HIV-infected subject. Also the invention may be worded as dietary fiber, or a composition comprising dietary fiber, for normalizing intestinal bacterial flora in a HIV-infected subject.

A “HIV-infected subject” is a person wherein according to commonly known criteria infection with HIV has been determined. Although such a subject may not show any disease symptoms or apparently may not be ill, such a subject may also be identified as a HIV-infected patient or HIV patient.

The term “normalization” as used herein in “normalization of intestinal bacterial flora”, refers to arriving at levels of intestinal bacterial flora that are on average found in healthy, non-HIV-infected subjects.

In one embodiment “normalization of intestinal bacterial flora” refers to stimulation of healthy gut flora (microbiota). In one embodiment “normalization of intestinal bacterial flora” refers to stimulation of, or an increase in, Bifidobacteria and/or lactobacilli. In one embodiment “normalization of intestinal bacterial flora” refers to a decrease of pathogenic bacteria. In one embodiment “normalization of intestinal bacterial flora” refers to a decrease of Pseudomonas.

The term “dietary fiber” as used in the present description, is meant to include indigestible oligosaccharide and indigestible polysaccharide.

Dietary fibers as used in this invention are typically resistant to digestion and absorption in the human small intestine with preferably a complete or partial fermentation in the large intestine. Preferably the present composition comprises at least one dietary fiber capable of stimulating the growth of bifodobacteria in the gut selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, pectin, pectate, chondroitine, hyaluronic acids, heparine, heparane, bacterial carbohydrates, sialoglycans, fucooligosaccharides, xanthan gum, polydextrose (PDX), galactomannans, preferably guar gum, arabinoxylan, preferably MGN-3 Rice Bran Arabinoxylan according to U.S. Pat. No. 5,560,914, xyloglycan, callose, and/or degradation products thereof. All of these have beneficial prebiotic and bifidogenic effects in the intestinal system. PDX is a non-digestible carbohydrate that has been synthesized from randomly cross-linked glucose and sorbitol.

As already mentioned, the finding that the intestinal flora in HIV-infected subjects is changed compared to healthy, non-HIV infected subjects led the inventors to the surprising finding that the known indigestible oligosaccharides such as galacto oligosaccharides (GOS) and fructooligosaccharides (inulin) are capable of stimulating the growth of Bifidobacteria in the HIV patients.

Table 1 shows clearly the effect of a composition comprising dietary fibers GOS and inulin on the content of Bifidobacteria in the feces as a percentage of the total fecal bacterial content. Before and after treatment with the dietary fibers there is a clear increase in the numbers of bifidobacteria.

The inventors surprisingly found that lactobacilli are also significantly decreased in the intestinal flora of HIV-infected subjects. This led the inventors to use a specific blend of oligosaccharides, known to stimulate the growth of lactobacilli and bifidobacteria in bottle-fed infants, comprising of a mixture that could not only improve the growth of Bifidobacteria but also the growth of lactobacilli in the intestines of HIV-infected subjects. The study resulted in a clear increase in lactobacilli in the feces of HIV-infected subjects that received a composition comprising galacto-oligosaccharides (GOS) and fructooligosaccharides (Inulin).

In a preferred embodiment the composition that is administered to HIV-infected subjects comprises at least two different oligosaccharides that stimulate both the growth of Bifidobacteria and lactobacilli. In one embodiment the composition comprises at least two different oligosaccharides selected from the group consisting of galactooligosaccharides including trans galactooligosaccharides, inulin, fructooligosaccharides, xylooligosaccharides, palati noseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, pectin, pectate, chondroitine, hyaluronic acids, heparine, heparane, bacterial carbohydrates, sialoglycans, fucooligosaccharides, xanthan gum, polydextrose (PDX), galactomannans and preferably guar gum, arabinoxylan, preferably MGN-3 Rice Bran Ara binoxylan according to U.S. Pat. No. 5,560,914, xyloglycan, callose, and/or degradation products thereof, wherein at least one is selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, xanthan gum, arabinoxylan, polydextrose (PDX), galactomannans, guar gum, and/or degradation products thereof. In one embodiment the two different oligossaccharides are galacto-oligosaccharides (GOS) and fructooligosaccharides (Inulin).

The dietary fibers are preferably administered in an amount of between 10 mg and 100 gram per day, preferably between 100 mg and 50 grams per day, even more between 1 and 25 gram per day. In one embodiment dietary fiber is administered in a daily dose of at least 10 g.

In order to have the most optimal prebiotic effect in HIV patients it is preferred the dietary fibers are all soluble, Further, in one embodiment of the present invention it is preferred the dietary fibers include indigestible oligosaccharide having an average degree of polymerisation (DP) of less than 15. It appears such short chain fibers are much more efficient in the stimulation of the growth of Bifidobacteria in the HIV patients.

In a preferred embodiment according to the present invention the dietary fiber that is used comprises at least two different indigestible oligosaccharides with a different average degree of polymerisation (DP) of at least a factor 2, preferably a factor 3 even more preferably a factor 4 and most preferably at least a factor 5 and the average degree of polymerisation of the dietary fiber with the lower DP is between 1-8. It has been shown that the combination of two dietary fibers with a factor 5 difference in average DP act synergistically in in vitro models for measuring prebiotic effects compared to two dietary fibers with an average DP that differ less than a factor 2.

In another preferred embodiment the weight ratio of the dietary fiber with the lower DP to the dietary fiber with the higher DP is between 1 and 9. It has been shown that in this range the prebiotic effect of the fibers is optimal.

Apart from the increased numbers of Bifidobacteria and lactobacilli, it is also required to eliminate as many as possible pathogenic bacteria from the intestinal environment of HIV-infected subjects in order to optimally stimulate the intestinal functions. HIV patients often suffer from diarrhea induced by pathogens leading to a decrease in the quality of life of these patients. Adherence of pathogenic bacteria is an important factor defining the pathogenicity of bacteria. In addition to the stimulation of the growth of Bifidobacteria and Lactobacilli it is thus desirable to inhibit the growth of pathogenic bacteria in the gut of HIV patients.

Therefore, in an even more preferred embodiment the composition that is administered to HIV-infected subjects comprises an additional dietary fiber selected from the group consisting of acid oligosaccharides. Without being bound by theory it is assumed that these acid oligosaccharides inhibit the adhesion of pathogenic bacteria to the intestinal mucosa.

The acid oligosaccharides used in the invention are preferably selected from the group consisting of pectin, pectate, alginate, chondroitine, hyaluronic acids, heparine, heparane, bacterial carbohydrates, sialoglycans, fucoidan, fucooligosaccharides and carrageenan and/or degradation products thereof, preferably selected from pectin and alginate, more preferably pectin or degradation products thereof. The acid oligosaccharides may be prepared by the methods described in WO 01/60378 or WO 02/042484, which are hereby incorporated by reference.

Alginates are linear unbranched polymers containing β-(1→4)-linked D-mannuronic acid and α-(1→4)-linked L-guluronic acid residues with a wide range of average molecular weights (100-100000 residues). Suitable sources of alginate include seaweeds and bacterial alginates.

Pectin is divided into two main categories: high methoxylated pectin, which is characterized by a degree of methoxylation above 50% and low methoxylated pectin having a degree of methoxylation below 50%. As used herein, “degree of methoxylation” (also referred to as DE or “degree of esterification”) is intended to mean the extent to which free carboxylic acid groups contained in the polygalacturonic acid chain have been esterified (e.g. by methylation). The present acid oligosaccharide is preferably prepared from high methoxylated pectin.

In one embodiment the acid oligosaccharides have a degree of methoxylation above 20%, preferably above 50% even more preferably above 70%. In one embodiment the acid oligosaccharides have a degree of methylation above 20%, preferably above 50% even more preferably above 70%.

The acid oligosaccharide is preferably administered in an amount of between 10 mg and 100 gram per day, preferably between 100 mg and 50 grams per day, even more between 0.5 and 20 gram per day.

Table 2 shows the relative number of Pseudomonas in the feces of HIV-infected subjects. It further shows the effect of a significant decrease of the oligosaccharide mixture on the intestinal Pseudomonas load in HIV-infected subjects before and after treatment with a fiber mixture comprising the acid oligosaccharide, here pectin hydrolysate.

In one embodiment the composition that is administered to HIV infected subjects comprises a dietary fiber that is selected from the group consisting of galactooligosaccharides including trans galactooligosaccharides, inulin, fructooligosaccharides, xylooligosaccharides, palati noseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, xanthan gum, arabinoxylan, polydextrose (PDX), galactomannans, guar gum, and/or degradation products thereof and an acid oligosaccharide dietary fiber that is selected from the group consisting of pectin, pectate, alginate, chondroitine, hyaluronic acids, heparine, heparane, bacterial carbohydrates, sialoglycans, fucoidan, fucooligosaccharides and carrageenan and/or degradation products thereof.

In one embodiment the composition that is administered to HIV infected subjects comprises at least two different dietary fiber that are selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, xanthan gum, arabinoxylan, polydextrose (PDX), galactomannans, guar gum, and/or degradation products thereof and an acid oligosaccharide dietary fiber that is selected from the group consisting of pectin, pectate, alginate, chondroitine, hyaluronic acids, heparine, heparane, bacterial carbohydrates, sialoglycans, fucoidan, fucooligosaccharides and carrageenan and/or degradation products thereof.

In one embodiment the composition that is administered to HIV infected subjects comprises galacto-oligosaccharides (GOS), fructooligosaccharides (Inulin) and pectin or degradation products thereof.

A completely different way of modulating the flora is by using nutritional ingredients comprising antibodies. The human immune system normally produces several forms of specific antibodies that are secreted in the intestinal mucosa. These antibodies prevent the adherence of potential pathogenic bacteria to the intestinal mucosa and thereby prevent the growth of these pathogens. HIV patients however, have a compromised immune system that becomes gradually insufficient to comply with this task. The oral supplementation with antibodies capable of preventing the adherence of potential pathogenic bacteria is therefore beneficial for HIV patients or other immune compromised patients e.g. cancer patients, malnourished or elderly.

Preferably bovine colostrum is used in the method and included in the composition according to the invention. Colostrum is naturally rich in antibodies against a plurality of potential pathogenic bacteria. Since antibodies in colostrum are easily destroyed during processing, great care should be applied in selecting the right commercially available colostrum. It is preferred that at least 25% of the total IgG antibodies present in the colostrum is undenatured, preferably at least 35%, more preferably between 60 and 100% and even more preferably between 70 and 100% is in undenatured form. In one embodiment colostrum is administered in a daily dose of at least 8 g. The use of colostrum will have as additional advantage over other immunoglobulin preparations, that it also comprises peptides and proteins beneficial for the barrier integrity. These are e.g. growth factors such as insulin like growth factor and transforming growth factor beta.

When the flora is changed by using the product it is has a significant effect on the barrier function of the gut and thereby decreasing the translocation of bacterial products over the intestinal barrier into the bloodstream. This eventually reduces the chronic activation of the immune system of HIV patients, detrimental to the progression of HIV to AIDS. Thus, any of the compositions according to the invention can be used for the treatment and prevention of chronic activation of the immune system in HIV patients.

A preferred composition suitable in the method of normalizing intestinal bacterial flora according to the invention comprises at least indigestible oligosaccharides and/or polysaccharides. Optionally probiotic bacteria or colostrum can be used to further enhance the prebiotic induced effects on the intestinal microflora and the chronic activation of the immune system during progressive HIV disease. A preferred composition comprises Bifidobacteria and/or lactobacilli as probiotic bacteria and/or bovine colostrum as preferred colostrum.

### Nutritional Compositions

The invention also concerns a nutritional composition for the stimulation of a healthy gut flora in HIV patients comprising dietary fiber, colostrum, fat, digestible carbohydrates, vitamins and minerals, wherein

a. the dietary fibers comprises of at least two different oligosaccharides selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, pectin, pectate, chondroitine, hyaluronic acids, heparine, heparane, bacterial carbohydrates, sialoglycans, fucooligosaccharides, xanthan gum, polydextrose (PDX), galactomannans and preferably guar gum, arabinoxylan, preferably MGN-3 Rice Bran Arabinoxylan according to U.S. Pat. No. 5,560,914, xyloglycan, callose, and/or degradation products thereof, wherein at least one is selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, xanthan gum, arabinoxylan, polydextrose (PDX), galactomannans, guar gum, and/or degradation products thereof, and

b. the colostrum comprises of at least 25 wt % undenatured IgG based on the total protein content of the colostrum, and

c. the fat content of the composition comprises between 10-30 en % of the total composition.

It is particularly advantageous that the nutritional composition comprises at least two different indigestible oligosaccharides with a different average degree of polymerisation (DP) of at least a factor 2, preferably a factor 3 even more preferably a factor 4 and most preferably at least a factor 5 and the average degree of polymerisation of the dietary fiber with the lower DP is between 1-8.

In one embodiment in the nutritional composition according to the invention at least two different dietary fibers are included that are selected from the group consisting of galactooligosaccharides including trans galactooligosaccharides, inulin, fructooligosaccharides, xylooligosaccharides, palati noseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, xanthan gum, arabinoxylan, polydextrose (PDX), galactomannans, guar gum, and/or degradation products thereof.

In one embodiment in the nutritional composition according to the invention galactooligosaccharides (GOS) and fructooligosaccharides (Inulin) are included.

Preferably the dietary fibers are present in an amount sufficient to stimulate the bifidobacteria and lactobacilli and at the same time to decrease the number of potential pathogenic bacteria in the intestinal flora of subjects with HIV.

In one embodiment therefore the nutritional composition according to the invention comprises a dietary fiber that is selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, xanthan gum, arabinoxylan, polydextrose (PDX), galactomannans, guar gum, and/or degradation products thereof and an acid oligosaccharide dietary fiber that is selected from the group consisting of pectin, pectate, alginate, chondroitine, hyaluronic acids, heparine, heparane, bacterial carbohydrates, sialoglycans, fucoidan, fucooligosaccharides and carrageenan and/or degradation products thereof.

In one embodiment the nutritional composition according to the invention comprises at least two different dietary fiber that are selected from the group consisting of galactooligosaccharides including trans galactooligosaccharides, inulin, fructooligosaccharides, xylooligosaccharides, palatinoseoligosaccharide, resistant starch, lactulose, lactosucrose, mannanoligosaccharides, isomaltooligosaccharides, maltooligosaccharides, glucomannan, arabinogalactan, soybean oligosaccharide, gentiooligosaccharide, xanthan gum, arabinoxylan, polydextrose (PDX), galactomannans, guar gum, and/or degradation products thereof and an acid oligosaccharide dietary fiber that is selected from the group consisting of pectin, pectate, alginate, chondroitine, hyaluronic acids, heparine, heparane, bacterial carbohydrates, sialoglycans, fucoidan, fucooligosaccharides and carrageenan and/or degradation products thereof.

In one embodiment in the nutritional composition according to the invention galactooligosaccharides (GOS) and fructooligosaccharides (Inulin) and pectin or degradation products thereof are included.

Dietary fibers and colostrum may be given as nutritional compositions comprising sufficient amount of fibers, e.g. oligosaccharides and/or colostrum to improve the bacterial flora. At least 1.5 gram of fiber per daily dose is required for obtaining the required effect. The best effect is obtained however with higher doses wherein at least 5 and preferably at least 10 gram fiber is delivered per daily dose. Preferably a mixture of fibers is given that stimulates both Bifidobacteria and lactobacilli. Even more preferably acid oligosaccharides are added to effectively prevent the adhesion and growth of pathogenic bacteria.

In a preferred embodiment the present composition comprises at least 2.5 gram bifidobacteria stimulating fibers, at least 2.5 gram acid oligosaccharides.

Preferably, the composition additionally comprises colostrum in an amount sufficient to provide at least 2 gram immunoglobulins per daily dose. If the colostrum that is used comprises more than 25 weight % undenatured immunoglobulin G based on the weight of total protein in colostrum powder it will have an additional effect on the improvement of the intestinal barrier function. A preferred embodiment therefore comprises at least 10 gram dietary fiber and at least 8 gram colostrum per daily dose.

The composition preferably is in the form of a drink, powder or bar and may further comprise additional protein, fat and vitamins and minerals. Preferably the present nutritional composition contains between 10 and 30 en % lipid, between 15 and 40 en % protein and between 25 and 75 en % carbohydrate (en % is short for energy percentage and represents the relative amount each constituent contributes to the total caloric value of the preparation).

In one embodiment the composition preferably is in liquid form and has a limited viscosity. Compositions containing dietary fibers including acid oligosaccharides, provide a liquid nutrition with sufficiently low viscosity so it can be applied as liquid clinical food which can be fed through a tube or a straw, while retaining the low viscosity. In a preferred embodiment, the present composition has a viscosity below 600 mPas, preferably below 250 mPas, more preferably below 50 mPas, most preferably below 25 mPas at a shear rate of 100 s⁻¹ at 20° C. Where the term viscosity is used in the present document, this refers to the physical parameter which is determined according to the following method:

The viscosity may be determined using a Carri-Med CSL rheometer. The used geometry is of conical shape (6 cm 2 deg acrylic cone) and the gap between plate and geometry is set on 55 μm. A linear continuous ramp shear rate is used from 0 to 150 s⁻¹ in 20 seconds.

The composition preferably comprises vegetable lipids as fat source. The vegetable lipid is preferably at least one selected from the group consisting of soy oil, palm oil, coconut oil, safflower oil, sunflower oil, corn oil, canola oil and lecithin. Animal fats such as milk fats or fish oils may also be added if desired. Preferably at least 15 en % of the total composition comprises of vegetable lipids and preferable no more than 30 en %. This is to prevent a too high energy load of the product that could easily compromise the palatability and compliance of the product.

The proteins used in the nutritional composition are preferably selected from the group of non-human animal proteins (such as milk proteins, meat proteins and egg proteins), vegetable proteins (such as soy protein, wheat protein, rice protein, and pea protein), free amino acids and mixtures thereof. Cow milk proteins such as casein and whey proteins are particularly preferred, since the amino acid composition of these protein sources is particularly beneficial for HIV patients.

A source of digestible carbohydrate may be added to the nutritional formula. It preferably provides about 30% to about 70% of the energy of the nutritional composition. Any suitable (source of) carbohydrate may be used, for example sucrose, lactose, glucose, fructose, corn syrup solids, and maltodextrins, and mixtures thereof.

### Use of the Compositions

The compositions according to the invention can beneficially be used for modulation of the intestinal flora of HIV patients in particular the stimulation of Bifidobacteria and lactobacilli in the intestinal flora of HIV patients. The compositions can further be used for the treatment and/or prevention of pathogenic infections of the intestines of HIV patients and for the treatment and/or prevention of chronic activation of the immune system during progressive HIV infection.

Thus in one embodiment the invention also concerns the use of a composition comprising dietary fiber, in particular indigestible oligosaccharides and/or indigestible polysaccharides for the manufacture of a medicament for the normalization of intestinal bacterial flora and in particular for the stimulation of the growth of bifidobacteria and/or lactobacilli in the intestinal tract of HIV-infected subjects preferably at least to levels present in healthy, non-HIV-infected subjects, and/or in particular for decreasing pathogenic bacteria, preferably Pseudomonas, in the intestinal tract of HIV-infected subjects preferably at least to levels present in healthy, non-HIV-infected subjects.

In one embodiment the Bifidobacteria are stimulated to a level of at least 5% of total intestinal bacteria. In one embodiment Pseudomonas is decreased to a level of less than 0.06% of total intestinal bacteria.

In one embodiment the invention concerns the use of a composition comprising dietary fiber, in particular indigestible oligosaccharides and/or indigestible polysaccharides and preferably comprising colostrum for the manufacture of a medicament for the prevention and/or treatment of infections caused by bacterial pathogens and/or yeasts in HIV patients. In one embodiment the bacterial infection is caused by at least one microorganism selected from the pathogens Pseudomonas and Candida.

In one embodiment the invention concerns the use of a composition comprising dietary fiber, in particular indigestible oligosaccharides and/or polysaccharides for the manufacture of a medicament for the treatment and prevention of chronic activation of the immune system during HIV infection.

### Examples

### Treatment Protocol of HIV Patients

### Design:

The study is an exploratory study, testing dose-response effect, was randomized, double-blind and placebo-controlled in parallel group design.

Study duration was 16 weeks from the Baseline visit for each patient, of which 12 weeks of supplementation. Four weeks after the end of the supplementation period a follow-up visit was performed, where all parameters were analyzed.

Visits were performed for screening, baseline, and 4, 12 and 16 weeks after baseline.

### Study Objective:

To determine tolerance, safety and to establish the effect on the intestinal flora of daily consumption for 12 weeks of an oral supplement in non-symptomatic HIV seropositive adults.

The test composition, i.c fiber product, is provided as a powder in individual sachets comprising GOS, FOS (inulin) and pectin hydrolysate.

### Study Population:

57 Adult (>18 yr), male and female HIV-1 infected adults, not on HAART were randomized into two groups:

| | |
|---|---|
| 1. Single dose | (N = 19) |
| 2. Placebo | (N = 19) |

### Assessments:

Stool samples are collected at baseline and after 12 weeks.

Compliance is assessed at each visit by counting the number of returned study product sachets and subject assessment of use of product will be made.

### Method for Measuring the Fecal Bacteria

The numbers of Bifidobacteria in feces of HIV patients was determined by FISH as described previously (Harmsen et al., J Pediatr Gastroenterol Nutr 30:61-7; Langendijk et al., Appl Environ Microbiol 61:3069-75). The results are shown in Table 1.

Table 1 shows that treatment of HIV patients with a composition according to the invention comprising dietary fiber resulted in a significant increase in the percentage of Bifidobacteria compared to total bacterial content in their feces in week 12 compared to start of the study (baseline) (FISH data), whereas the control group did not show any significant change after 12 weeks of intake of the Placebo product.

TABLE 1
Fecal bifidobacteria (FISH data) in HIV
patients given as % of total bacteria

| Group | % bifidobacteria |
|---|---|
| Placebo (t = 0) | 2.0 |
| Placebo (t = 12 weeks) | 2.5 |
| Dietary fiber (t = 0) | 2.5 |
| Dietary fiber (t = 12 weeks) | 8.0 |

The numbers of Pseudomonas aeruginosa were determined by a 5′ nuclease assay based on Pirnay et al Crit Care 4:255-61. Briefly, a mixture of 20 μl containing 900 nM primers (F_pseudo, 5′-AACAGCGGTGCCGTTGAC-3′; R_pseudo, 5′-GTCGGAGCTGTCGTACTCGAA-3′; Biolegio, Nijmegen, The Netherlands), 200 nM probe (P_pseudo, VIC-5′-CGTGCGATCACCACC-3′-MGB-NFQ; Applied Biosystems, Nieuwerkerk aan de IJssel, The Netherlands), 1× TaqMan Fast Universal Master Mix (Applied Biosystems, Nieuwerkerk aan de IJssel, The Netherlands), milliQ and DNA is prepared. This mixture is subsequently used to run the 5′nuclease assay on the ABI 7700HT Fast (Applied Biosystems, Nieuwerkerk aan de IJssel, The Netherlands) with a temperature profile that consists of 20 seconds at 95° C. and 45 cycles of 1 second at 95° C. and 20 seconds at 60° C.

The Pseudomonas content in relation to the eubacterial content, which was determined according to Nadkarni et al Microbiology 148:257-66, was thereafter calculated as described earlier (Liu and Saint. 2002, Anal Biochem 302:52-9; Liu and Saint 2002, Biochem Biophys Res Commun 294:347-53). The results are shown in Table 2.

Table 2 shows that upon treatment of HIV patients with a composition according to the invention comprising dietary fiber a clear decrease in Pseudomonas content in the feces of the HIV was found indicating that the fibers are capable of reducing the number of potential pathogenic bacteria to manifest themselves in the intestinal tract of HIV patients.

TABLE 2
Fecal Pseudomonas in HIV patients given as % of total bacteria

| Group | % Pseudomonas |
|---|---|
| Placebo (t = 0) | 0.1 |
| Placebo (t = 12 weeks) | 0.08 |
| Dietary fiber (t = 0) | 0.08 |
| Dietary fiber (t = 12 weeks) | 0.02 |

These results show that the early impairment of the microbiota at the GI level in HIV positive patients is present since the very first phases of chronic HIV disease. This impairment is associated with heightened levels of intestinal inflammatory parameters, thus confirming the correlation between intestinal microbial alteration, GI mucosal damage, and immune activation status. Combined, these findings highlight a deep alteration of the mucosal barrier and strongly support the hypothesis put forward by Brenchely in which the injury at the GI tract level may represent a key factor in the pathogenesis of chronic HIV infection by sustaining the persistent immune activation associated with the progressive CD4+ cell depletion.

Preferred Composition for the treatment or maintenance
of a healthy or normal intestinal flora

| Raw Material | g/day | protein | carbs | fat |
|---|---|---|---|---|
| Colostrum | 20 | 15 | 2 | 0.8 |
| Galacto-oligosaccharides | 15 | 0 | 5 | 0 |
| Fructooligosaccharide | 0.79 | 0 | 0 | 0 |
| (Inulin) | | | | |
| Pectin hydrolysate | 8 | 0.1 | 0.1 | 0 |
| Fructose syrup | 15 | 0 | 12 | 0 |

| | |
|---|---|
| Vitamin A-E | according to Food for Special Medical |
| | Purposes regulations |
| Minerals | according to Food for Special Medical |
| | Purposes regulations |

1. A method for normalizing intestinal flora of an HIV-infected subject, comprising feeding the subject with a composition comprising dietary fiber, resulting in decreased amounts of pathogenic bacteria in said subject, thereby normalizing said intestinal flora.

2. The method according to claim 1, wherein the feeding stimulates growth of Bifidobacteria and/or Lactobacilli in the intestinal tract of said HIV-infected subject.

3. The method according to claim 1, wherein the amount of Pseudomonas bacteria in the intestinal flora of said HIV-infected subject is decreased.

4. The method according to claim 1, wherein the composition comprises at least galactooligosaccharides and inulin.

5. The method according to claim 3, wherein the composition comprises an acid oligosaccharide.

6. The method according to claim 5 wherein the composition comprises at least galactooligosaccharides, inulin and pectin or a degradation product of pectin.

7. The method according to claim 1 wherein at least 10 g dietary fiber are fed daily to the subject.

8. The method according to claim 1 wherein the composition further comprises colostrum.

9. The method according to claim 8 wherein the composition comprises at least 8 g colostrum in a daily dose.

10. A method for preventing and/or treating a Pseudomonas and/or Candida infection in an HIV-infected subject, comprising feeding the subject a composition comprising dietary fibers,
thereby preventing and/or treating said infection.

11. A method for treating or preventing a condition of chronic activation of the immune system in an HIV-infected subject, comprising feeding the subject a composition comprising dietary fibers,
thereby preventing or treating said condition.

12. A nutritional composition for the stimulation of healthy gut flora in an HIV-infected subject, comprising dietary fibers, colostrum, fat, digestible carbohydrates, vitamins and minerals, wherein:
(a) the dietary fibers comprise at least:
(i) galactooligosaccharides with an average degree of polymerization between 1 and 8, and
(ii) inulin with an average degree of polymerization of at least twice that of the average degree of polymerization of said oligosaccharides,
(b) the colostrum comprises of at least 25 wt % undenatured IgG based on total protein content of the colostrum, and
(c) the fat comprises between 10-30 en % of the total composition.

13. The nutritional composition according to claim 12 wherein the dietary fibers further comprise pectin or degradation products of pectin.

14. The nutritional composition according to claim 12 wherein the composition further comprises probiotic bacteria.

15. The method according to claim 3, wherein the composition comprises an acid oligosaccharide.

16. The method according to claim 15, wherein the acid oligosaccharide is pectin or a degradation product of pectin.
